(19) United States
(12) Patent Application Publication
(10) Pub. No.: US 2012/0178919 A1
(43) Pub. Date: Jul. 12, 2012

(54) RADIOLABELED NUCLEOSIDE ANALOGUE, AND PREPARATION METHOD AND USE THEREOF

(75) Inventors: HSIN-ELL WANG, Taipei city (TW); CHIH-YUAN LIN, Taoyuan County (TW); WEI-TI KUO, Taoyuan County (TW); CHUAN-LIN CHEN, Taipei City (TW); MEI-HUI WANG, Taoyuan County (TW); HUNG-MAN YU, Taoyuan County (TW); MAO-CHI WENG, Taoyuan County (TW); YU CHANG, Taipei City (TW); WUU-JYH LIN, Taoyuan County (TW)

(73) Assignees: NATIONAL YANG-MING UNIVERSITY, Taipei city (TW); Institute of Nuclear Energy Research Atomic Energy Council, Executive Yuan, Taoyuan County, FL (US)

(21) Appl. No.: 13/194,247

(22) Filed: Jul. 29, 2011

(30) Foreign Application Priority Data
Jan. 7, 2011 (TW) 100100579

(51) Int. Cl. C07H 19/067; C07H 19/09; C07H 19/073

(52) U.S. Cl. 536/28.5; 536/28.54

(57) ABSTRACT

A radiolabeled nucleoside analogue is provided, which includes radioactive iodine ¹²³I/¹³¹I, and a nucleoside analogue selected from a group consisting of cytidine, thymidine, uridine, and a derivative thereof. A method for preparing the radiolabeled nucleoside analogue, and a use thereof are further provided. The nucleoside analogue, prepared through the preparation method with a short synthesis time and a high radiochemical yield, has a long in vivo physiological half life and a high stability in serum, and, as a radiopharmaceutical composition, is useful in development of tumor proliferation diagnosis or therapy prognosis evaluation, and further assists in observation of long-time in vivo metabolism of a drug.

### BACKGROUND OF THE INVENTION

1. Field of Invention

The present invention relates to a radiolabeled nucleoside analogue, and particularly to a nucleoside analogue useful in imaging of tumor proliferation.

2. Related Art

Cancers have become the first leading cause of death in the world. Radiolabeled nucleoside analogue, in combination with positron emission tomography (PET) or single photon emission computed tomography (SPECT), can assist in clinical detection of tumor focus.

In proliferation of a malignant tumor, cell division is a necessary process, in which large quantities of deoxyribonucleic acid (DNA) sequences are generated, and precursors for forming a DNA sequence are nucleotides. Nucleosides are bonded with three phosphate groups in vivo in the presence of phosphorylase kinase, and then have a capability to be incorporated into the DNA sequence. The malignant tissue captures nucleotides at large quantity for division and proliferation.

DNA synthesis mainly performed through two pathways, a first pathway is a de novo pathway, in which nucleotide thymidine monophosphate (TMP) is formed by methylating deoxyuridine monophosphate (dUMP) in the presence of thymidylate synthase (TS); and the other pathway is a salvage pathway, in which exterior thymidine is ingested directly, and then bonded with three phosphate groups in the presence of thymidine kinase 1 (TK1), to form TMP. However, the precursor dUMP (derived from deoxyuridine, uridine, and uracil) used in the de novo pathway is also involved in the synthesis of RNA, thus being unsuitable for monitoring of the DNA synthesis. Therefore, researches are still mainly focused on nucleoside analogue in the salvage pathway as a tracer for detecting DNA synthesis at present.

In the salvage pathway, a critical enzyme is TK1, and it is pointed out in a reference that, the expression level of TK1 is closely related to cell cycle, which is high in G1 phase to S phase transition, but low in G0 or G1 phase (Sherley J L and Kelly T J. Regulation of human thymidine kinase during the cell cycle. J Biol Chem 1988;263:8350-8.). To sum up, TK1 level in tumor cells is higher than that in common normal cells (Schwartz J L, Tamura Y, Jordan R, Grierson J R, and Krohn K A. Monitoring tumor cell proliferation by targeting DNA synthetic processes with thymidine and thymidine analogs. J Nucl Med 2003; 44:2027-32.). Numerous nucleoside analogue probes have been developed at present, which are useful as contrast media in nuclear medicine according to the above mechanism Hereinafter, several existing nucleoside analogues for PET and SPECT are described and compared.

[¹¹C]thymidine ([¹¹C]TdR):

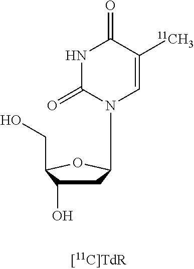

Radioisotope C-11 labeled thymidine [¹¹C] TdR is a first nucleoside radiopharmaceutical as a contrast medium in imaging of tumor proliferation rate (Christman D, Crawford E J, Friedkin M, and Wolf A P. Detection of DNA synthesis in intact organisms with positron-emitting (methyl-11 C) thymidine. Proc Natl Acad Sci USA 1972; 69: 988-92.). As [¹¹C]TdR has the same structure as that of natural thymidine, [¹¹C]TdR has the identical capability of being incorporated into DNA as that of natural thymidine, and the accumulation degree in the cells is proportional to the DNA synthesis rate, and thus the proliferation rate of tumor and normal tissues can be directly evaluated through quantitative analysis (Eary J F, Mankoff D A, Spence A M, Berger M S, Olshen A, Link J M, et al. 2-[C-11]thymidine imaging of malignant brain tumors. Cancer Res 1999;59:615-21.). However, due to the limitation of short physical half life (20 min) of C-11, clinical use of C-11 labeled radiopharmaceutical is limited, and [¹¹C]TdR is highly easily enzymatically cleaved in an organism, and thus the stability in the organism is poor (Shields A F, Lim K, Grierson J, Link J, and Krohn K A. Utilization of labeled thymidine in DNA synthesis: studies for PET. J Nucl Med 1990;31:337-42.). Therefore, [¹¹C]TdR is unsuitable as a contrast medium for imaging of tumor proliferation.

3′-Deoxy-3′-[¹⁸F]fluorothymidine ([¹⁸F]FLT):

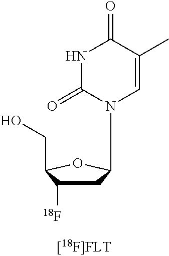

[¹⁸F]FLT is also a TdR analogue, and is one of the most commonly used tracers in evaluation of proliferation rate of tumor and normal tissues, and the efficacy has been verified by multiple tumor patterns, for examples, long cancer, colorectal cancer, and lymphoma (Francis D L, Visvikis D, Costa D C, Arulampalam T H, Townsend C, Luthra S K, et al. Potential impact of [¹⁸F]3′-deoxy-3′-fluorothymidine versus [¹⁸F]fluoro-2-deoxy-D-glucose in positron emission tomography for colorectal cancer. Eur J Nucl Med Mol Imaging 2003;30:988-94; Seitz U, Wagner M, Neumaier B, Wawra E, Glatting G, Leder G, et al. Evaluation of pyrimidine metabolising enzymes and in vitro uptake of 3′-[¹⁸F]fluoro-3′-deoxythymidine ([¹⁸F]FLT) in pancreatic cancer cell lines. Eur J Nucl Med Mol Imaging 2002; 29:1174-81; Vesselle H, Grierson J, Muzi M, Pugsley J M, Schmidt R A, Rabinowitz P, et al. In vivo validation of 3′deoxy-3′-[¹⁸F] fluoro thymidine ([¹⁸F]FLT) as a proliferation imaging tracer in humans: correlation of [¹⁸F]FLT uptake by positron emission tomography with Ki-67 immunohistochemistry and flow cytometry in human lung tumors. Clin Cancer Res 2002;8:3315-23; Buck A K, Schirrmeister H, Hetzel M, Von Der Heide M, Halter G, Glatting G, et al. 3-deoxy-[¹⁸F]fluorothymidine-positron emission tomography for noninvasive assessment of proliferation in pulmonary nodules. Cancer Res 2002;62:3331-4; Dittmann H, Dohmen B M, Kehlbach R, Bartusek G, Pritzkow M, Sarbia M, et al. Early changes in [¹⁸F]FLT uptake after chemotherapy: an experimental study. Eur J Nucl Med Mol Imaging 2002; 29:1462-9; Vijayalakshmi D and Belt J A. Sodium-dependent nucleoside transport in mouse intestinal epithelial cells. Two transport systems with differing substrate specificities. J Biol Chem 1988;263:19419-23.). F-18 is a radionuclide capable of emitting positron, and having a suitable half life of 110 min, and can mimic hydrogen in nature since the Vander Waals radius is similar to that of a hydrogen atom, thus being a radionuclide applicable in molecular imaging in nuclear medicine. As an OH group originally existing on carbon 3′ of a glycosyl group is substituted with F-18 atom, [¹⁸F]FLT is provided with the capability of countering nucleoside phosphorylase to cleave a N-glycosidic bond; however, the position of the OH group originally recognized by DNA polymerase for extension of DNA sequence is altered for the same reason, such that FLT is phosphated by TK1 and remained in the cells, but cannot be further incorporated into the DNA sequence. Therefore, the accumulation of FLT in a tissue merely indicates in a biological sense that the TK1 activity in the tissue is high (if the cell is in an S phase, TK1 level is relatively high), and does not absolutely directly correlate to the proliferation rate. Therefore, PET imaging of [¹⁸F]FLT can reflect the thymidine demand of tumor cells and TK1 activity, and thus the proliferation rate of tumor cells can be indirectly obtained.

2-[¹⁸F]fluoro-5-methyl-1-β-D-arabinofuranosyluracil ([¹⁸F]FMAU):

In view of the problems existing in use of [¹¹C] thymidine and [¹⁸F]FLT, specialists in the field are driven to find other promising contrast media for imaging of tumor proliferation rate. It is pointed out in previous researches that, a hydrogen atom at position 2′ of the glycosyl group in thymidine is substituted with F-18 in [¹⁸F]FMAU, such that nucleoside phosphorylase is blocked from breaking off of the N-Glycosidic bond in an organism Therefore, [¹⁸F]FMAU is very stable in the organism, and like TdR, can be incorporated in a DNA sequence in the DNA synthesis phase (S phase) of a cell in presence of an enzyme in the organism, and thus the accumulation degree of [¹⁸F]FMAU in a cell is considered to be proportional to the DNA generation rate and the cell proliferation rate. However, the synthesis of [¹⁸F]FMAU marker requires a long period of time, and the radiochemical yield is low (Namavari M, Barrio J R, Toyokuni T, Gambhir S S, Cherry S R, Herschman H R, et al. Synthesis of [¹⁸F]fluoroguanine derivatives: in vivo probes for imaging gene expression with positron emission tomography. Nucl Med Biol 2000;27:157-62.).

5-[^{124/131}I]iodo-2′-deoxyuridine ([^{124/131}I]IUdR)

[^{124/131}I]IUdR is also a TdR analogue, in which an original methyl group at position 5 of the phenyl ring is substituted by iodine, and the design principle for the chemical structure is that iodine has a Vander Waals radius similar to that of methyl at the carbon atom of position 5 of thymidine. IUdR can be incorporated into DNA in mitosis of a cell, and thus the accumulation of IUdR in a tissue of an organism directly positively correlates to the cell proliferation rate. In recent years, studies on treatment of malignant tumors with [¹²⁵I]IudR are reported in literatures; however, due to the quite short in vivo physiological half life of IUdR (5 min in human body and 7 min in mice, as shown in literatures) (Prusoff W H. A Review of Some Aspects of 5-Iododeoxyuridine and Azauridine. Cancer Res 1963;23:1246-59.), use of [^{124/131}I]IUdR in imaging of tumors is limited even if radioactive iodine with a long physical half life is used.

### SUMMARY OF THE INVENTION

In view of the disadvantages of nucleoside analogues for imaging, it is necessary to develop a novel nucleoside analogue useful in single photon emission computed tomography (SPECT) of tumor.

The present invention is directed to a radiolabeled nucleoside analogue, which has a long in vivo physiological half life and a high stability in serum.

The present invention is further directed to a method for preparing the radiolabeled nucleoside analogue with a short synthesis time and a high radiochemical yield.

The present invention is further directed to a use of the radiolabeled nucleoside analogue, as a radiopharmaceutical composition, which has a high specificity, a short synthesis time, a high radiochemical yield, and a long half life, and is useful in development of tumor proliferation diagnosis or therapy prognosis evaluation, and further assists in observation of long-time in vivo metabolism of a drug

In order to achieve the above objectives, the present invention provides a radiolabeled nucleoside analogue, comprising a compound having a chemical formula below:

A—B

in which, A is radioactive iodine comprising ¹²³I, ¹³¹I, and ¹²⁴I, and B is a pyrimidine derivative selected from a group consisting of cytidine, thymidine, uridine, and a derivative thereof

In the radiolabeled nucleoside analogue, the pyrimidine derivative is cytidine or thymidine. The pyrimidine derivative is a pyrimidine derivative comprising 1-(2-deoxy-β-D-arabinofuranosyl)-5-tributylstannyl. By using the characteristic that the TK1 level in tumor cells is higher than that in normal cells, proliferation rates of tumor and normal tissues are directly or indirectly evaluated through quantitative analysis of accumulation degree of the radiolabeled nucleoside analogue and DNA synthesis rate in the cells by monitoring DNA synthesis in the tumor cells.

As for the radioactive iodine, the decay mode of ¹²³I is electron capture in which γ rays and Auger electrons are emitted, and the half life is about 13.2 hours, and the decay mode of ¹³¹I is β decay in which γ rays and β particulates are emitted, and the half life is about 7-8 days. Therefore, large quantities of Auger electrons emitted by ¹²³I may be used to effectively cause local double strand break of DNA, thus resulting in death of tumor cells. The destructive power of ¹³¹I to tumor cells is not as high as that of ¹²³I, ¹³¹I can emit β particulates with βₘₐₓ of 606 keV and rays, and has a wide effective kill range and a diagnosis function through imaging. Therefore, on one hand, the tumor position can be accurately determined according to the radioactive tracing property, and the tumor cells can be killed with the emitted rays one the other hand. Optionally, radioactive iodine may be replaced by other radionuclides, such as ^{99m}Tc or ¹¹¹In, and in case of positron emission tomography (PET), radionuclide ¹²⁴I may be used.

The present invention also provides a method for preparing radiolabeled nucleoside analogue, comprising:

(a) preparing a labeling precursor comprising 5-tributylstannyl-2′-pyrimidine derivative, in which the pyrimidine derivative in the labeling precursor is selected from a group consisting of cytidine, thymidine, uridine, and a derivative thereof;

(b) iododestannylating the labeling precursor with a radionuclide under oxidation conditions, to obtain radioactive iodine labeled crude product, in which the radioactive iodine comprises ¹²³I and ¹³¹I; and

(c) purifying the radioactive iodine labeled crude product, to obtain the radiolabeled nucleoside analogue.

In the preparation method, the pyrimidine derivative in Step (a) is cytidine or thymidine. More specifically, the labeling precursor is a pyrimidine derivative comprising 1-(2-β-D-arabinofuranosyl)-5-tributylstannyl.

In the preparation method, the oxidation condition in Step (b) is oxidation with hydrogen peroxide.

In the preparation method, the purification in Step (c) is performed on a silica gel column, and the purified radiolabeled nucleoside analogue may exist in a form of lyophilized powder.

The present invention further provides a radiopharmaceutical composition, comprising the radiolabeled nucleoside analogue. The radiolabeled nucleoside analogue comprises a compound having a Structural Formula I below,

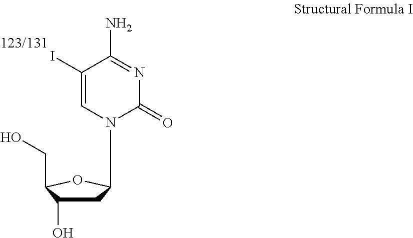

or a compound having a Structural Formula II below.

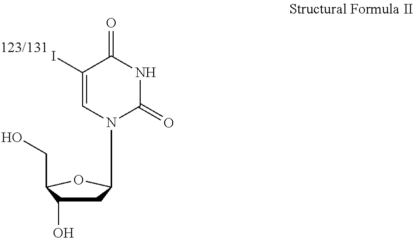

The radiopharmaceutical composition of the present invention is useful as a contrast medium for imaging of tumor proliferation, and assists in development of imaging in nuclear medicine in tumor detection or therapy prognosis evaluation, rays emitted by the radiopharmaceutical composition can also be used in treatment of malignant tumor, to effectively inhibit the regeneration of malignant tumor, and the radiopharmaceutical composition and the emitted rays can further be used in combination, so as to achieve the dual purpose of diagnosis through imaging and treatment in nuclear medicine.

### BRIEF DESCRIPTION OF THE DRAWINGS

The present invention will become more fully understood from the detailed description given herein below for illustration only, and thus are not limitative of the present invention, and wherein:

FIG. 1 shows experimental results of reversed thin-layer chromatography of ^{[123/131}I]ICdR in Example 3 according to a preferred embodiment of the present invention;

FIG. 2 shows experimental results of reversed thin-layer chromatography of ^{[123/131}I]IUdR in Example 3 according to a preferred embodiment of the present invention;

FIG. 3 is a high performance liquid chromatography (HPLC) diagram of standard ICdR and [¹³¹I]ICdR in Example 3 according to a preferred embodiment of the present invention;

FIG. 4 shows uptake test data (a) and regression analysis results (b) obtained by adding two radiolabeled nucleoside analogues [¹³¹I]IUdR and [¹³¹I]ICdR to cells in Example 4 according to a preferred embodiment of the present invention;

FIG. 5 shows DNA extraction experimental results in Example 5 according to a preferred embodiment of the present invention, which shows that ¹³¹I-ICdR (A) and ¹³¹I-IUdR (B) are linearly incorporated into NG4TL4 sarcoma cells with time, and found to be radioactively accumulated in DNA;

FIG. 6 shows blood activity variation results obtained through regular blood withdrawal after two radioactive nucleoside analogues [¹³¹I]ICdR (a) and [¹³¹I]IUdR (b) are intravenously injected into mice in Example 7 according to a preferred embodiment of the present invention (n=3 at each time point);

FIG. 7 shows results of planar ₇ imaging and animal micro-SPECT/CT imaging in Example 9 according to a preferred embodiment of the present invention, obtained by injecting ¹²³I-ICdR (A), ¹²³I-IUdR (B), and ¹²³I-ICdR (C) respectively into mice with NG4TL4 sarcoma (arrow) (n=4); and

FIG. 8 shows results of planar γ imaging and animal micro-SPECT/CT imaging in Example 9 according to a preferred embodiment of the present invention, which is obtained by injecting ¹³¹ICdR (A), ¹³¹I-IUdR (B), and ¹²³I-ICdR (C) respectively into mice implanted with malignant LL/2 lung sarcoma (arrow) (n=4).

### DETAILED DESCRIPTION OF THE INVENTION

Hereinafter, embodiments of the present invention are described in detail with reference to examples below; however, the present invention is not limited thereto.

### EXAMPLE 1

### Synthesis of Standard 5-iodo-2′-deoxycytidine (ICdR)

As the commercially available starting material deoxycytidine hydrochloride is sparsely soluble in methanol, and thus before reaction, deoxycytidine hydrochloride (1 g) was dissolved in methanol (2 mL) first, and then several drops of triethylamine were added for neutralization, till deoxycytidine hydrochloride was completely dissolved. The solution was added to a sample vial containing CH₂Cl₂, and then large quantities of precipitate were generated, which was filtrated to obtain neutralized deoxycytidine as a solid (as shown in Formula 1).

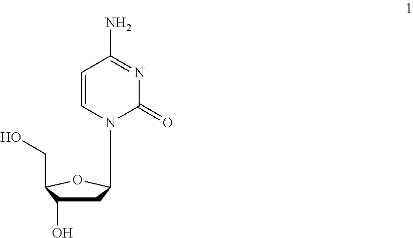

A rotor was added to a 25 mL round-bottom flask, and then 0.4 g deoxycytidine was dissolved in 30 mL methanol, and stirred for a few minutes. Iodine (670 mg, 1.5 eq) and silver trifluoroacetate (583 mg, 1.5 eq) were added in sequence, and reacted for about 20 hours at 35° C., and a precipitate silver iodide was generated. After reaction, the reaction solution was filtrated with celite, and washed with methanol, and the filtrate was dried by suction. The product was purified by chromatography on silica gel column (eluting with dichloromethane/methanol=4/1 as a mobile phase), to obtain the following final product ICdR (as shown in Formula 2 below, 370 mg, yield: about 60%).

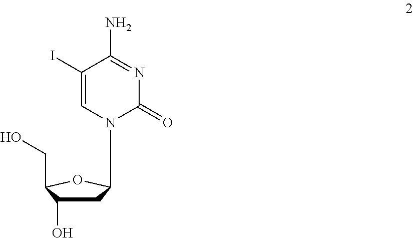

The chemical structure was identified by nuclear magnetic resonance (NMR) spectrum, and the data was as follows.

¹H NMR (MeOH-d₄, 200 MHz): δ 8.43 (s, 1H, H-6), 6.08 (dd, J=6.0, 6.2 Hz, 1H, H-1′), 4.26 (m, 1H, H-3′), 3.77 (m, 3H, H-4′, H-5′), 2.23 (m, 1H, H-2′α), 2.05 (m, 1H, H-2′(3)

LRESI(+): 376.0 ([M+Na]⁺); Exact mass (HRMS) calcd for C₉H₁₂IN₃O₄, 352.9872; found 353.9959 ([M+H]⁺); found 375.9780 ([M+Na]⁺)

### EXAMPLE 2

### Synthesis of Labeling Precursor 1-(2-deoxy-β-D-arabinofuranosyl)-5-tributylstannyl cytosine (Bu₃SnCdR, as shown in Formula 3) of [¹³¹I]ICdR

200 mg (0.56 mmole) standard IcdR was placed in a flask, then 15.5 mg tris(dibenzylideneacetone)palladium (0) (0.03 eq, 0.00425 mmole) was added, and the system was evacuated, and filled with argon, so as to maintain the system in an argon atmosphere. 700 μL bis(tributyltin) (3.5 eq 1.4 mmol, d=1.158, MW=580.08) and then 2 mL dry DMF were added, and reacted overnight by heating to 65° C. in an oil bath. After reaction, the solution was filtrated with celite, dissolved in dichloromethane, and dried by suction, and the product was purified by chromatography on a silica gel column (eluting with CH₂Cl₂/MeOH=10/1 as a mobile phase), to obtain the final product Bu₃SnCdR as shown in Formula 3 (yield 40%). The method for packaging into sample vials includes dissolving 1.6 mg purified Bu₃SnCdR into 2 mL dry dichloromethane, then respectively injecting into vials (50 μL/kit), dried by suction under vacuum, filling with nitrogen, and capped, to complete the preparation of sample vials (40 μg/vial) of Bu₃SnCdR, which were stored in dark in an oxygen free environment.

¹H-NMR (CDCl₃, 400 MHz): δ 7.52 (s, 1H, H-6′), 6.06 (dd, J=6.0 Hz, 6.4 Hz, 1H, H-1′), 4.60 (s, 1H, H-3′), 4.05 (s, 1H, H-4′), 3.83(s, 2H, H-5′), 2.45 (s, 2H, H-2′), 0.84˜1.61 (m, 27H, SnBu₃)

LRESI(−): 516.5 ([M-H]⁻); Exact mass (HRMS) calcd for C₂₁H₃₉N₃O₄Sn, 517.1963; found 518.2079 ([M+H]⁺)

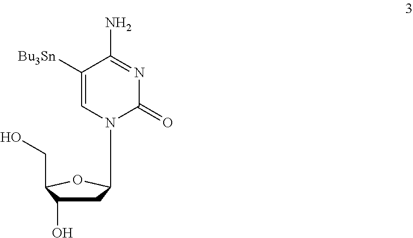

### EXAMPLE 3

### Synthesis of Radioactive ¹²³I and ¹³¹I labeled [^{123/131}I]IcdR and [^{123/31}I]IUdR

20 μL ethanol was respectively added into one sample vial (40 μg) of Bu₃SnUdR and Bu₃SnCdR to dissolve the drug. Suitable amount of [^{123/131}I]NaI solution and 100 μL solution of H₂O₂/HCl/H₂O=8/8/84 were added in sequence as oxidants and sealed by capping, and radioactivity was measured, followed by reaction for 10 min with vigorous shaking Then, the reaction mixture was directly cooled and solidified with liquid nitrogen, and active carbon tube was disposed, and the reaction mixture was placed in a vacuum system provided with active carbon adsorbent, for freezing drying, to remove unreacted radioactive iodine, the acid (HCl), the solvents (EtOH,H₂O), and the oxidant (H₂O₂), so as to obtain a freezing dried powder merely containing [^{123/131}]ICdR and trace CdR.

[^{123/131}I]IUdR was prepared through the same process, and was measured for radioactivity, and the labeling yield was obtained by comparing the radioactivity before and after reaction. Finally, suitable amount of saline was added to dissolve the product for reversed thin-layer chromatography (TLC).

In general, a scheme for synthesizing standard and labeled ICdR is as follows.

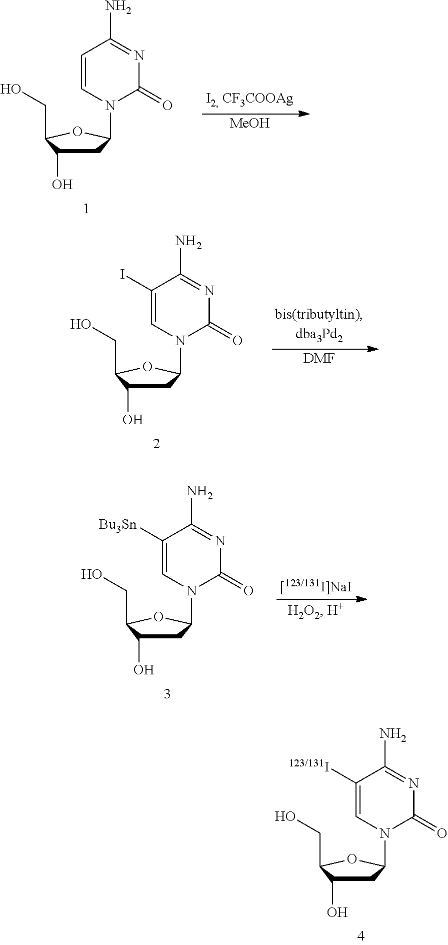

A scheme for synthesizing standard and labeled IUdR is as follows.

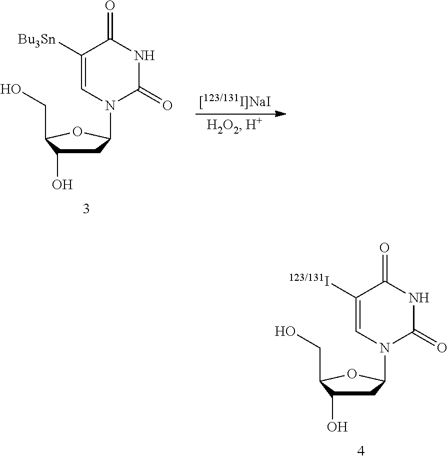

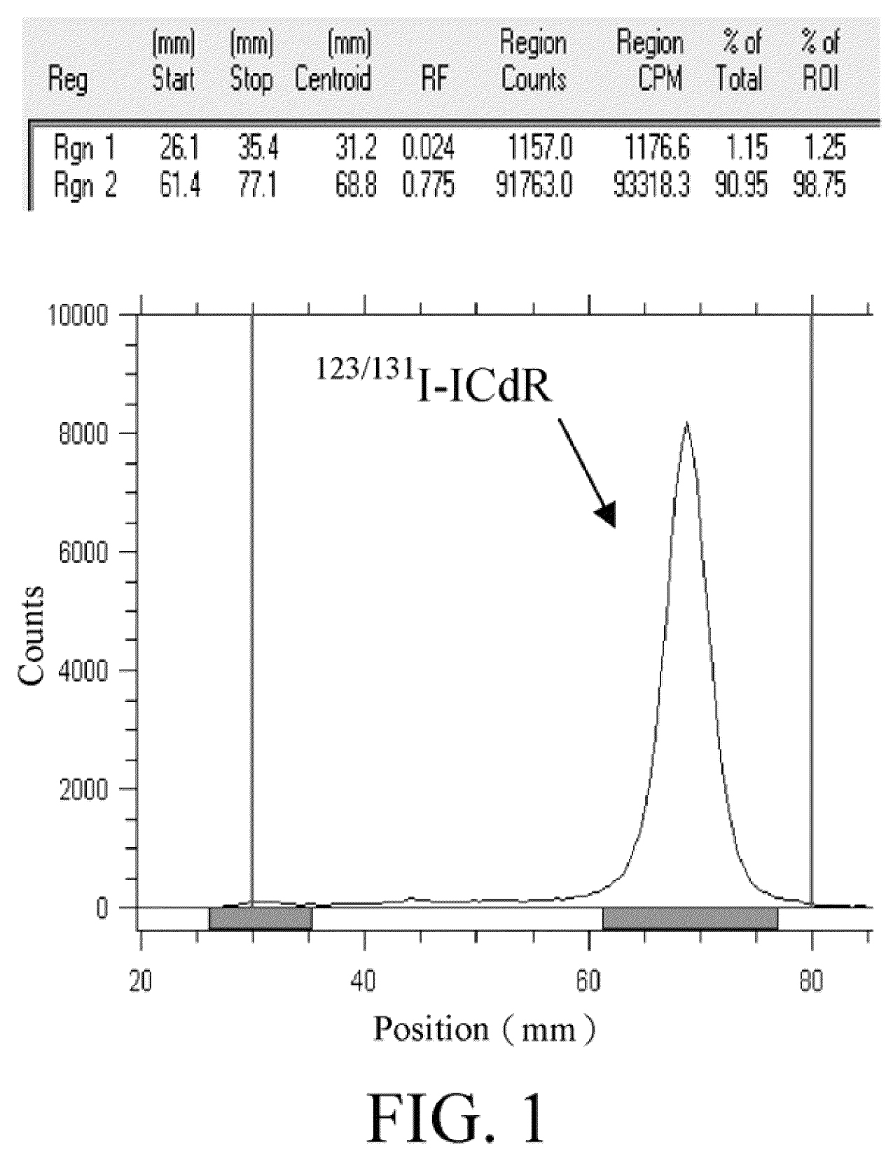

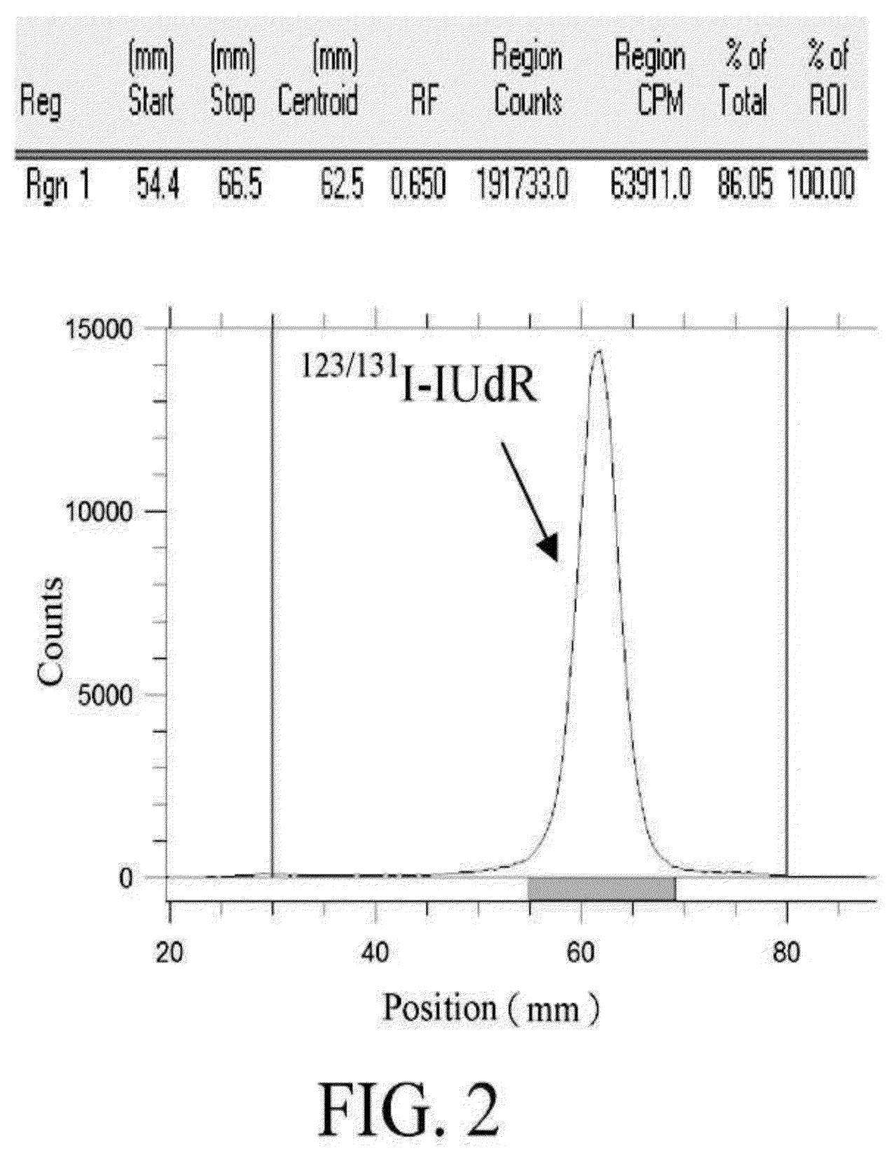

Experimental results of reversed TLC of [^{123/131}I]ICdR and [^{123/131}I]IUdR are respectively as shown in FIGS. 1 and 2. Radio TLC conditions: reversed TLC, developing solution 10 mM acetic acid/EtOH=2/1, and R_{f} values of [^{123/131}I]ICdR and [^{123/131}I]NaI are respectively 0.78 and 0.99. Developing solution of normal TLC is ethyl acetate/ethanol=5/1, and R_{f} value of [^{123/131}I]IUdR is 0.65.

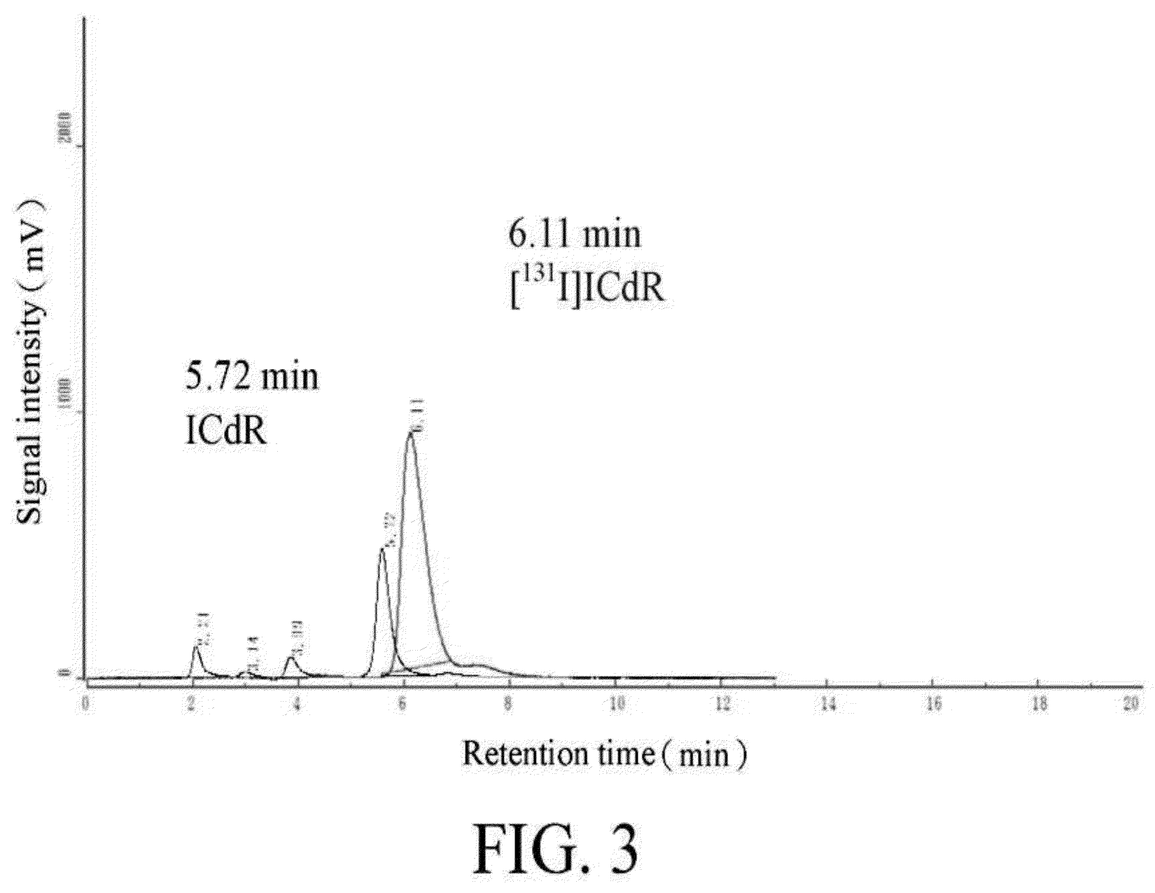

HPLC diagram of standard ICdR and [¹³¹I]ICdR are as shown in FIG. 3 (in which the developing phase is 10% acetonitrile and 90% 0.1% acetic acid, flow rate: 0.8 mL/min, analytical C18 column).

Biological property analysis of [^{123/131}I]ICdR and [^{123/131}I]IUdR are described below.

### EXAMPLE 4

### Cellular Uptake Test

2×10⁶ cells was inoculated in a 15 cm² dish containing 14 mL medium supplemented with 10% FBS. After 48-h incubation, the medium was replaced with a serum free medium containing radioactive tracers ¹³¹I-ICdR and ¹³¹I-IUdR (0.5˜1 μCi/mL medium). At specified time points (using I-131 tracer at 1, 2, 4 and 8 h), the cells on the dish was harvested by using a cell scraper. Then, a cell suspension was transferred to a 15 mL centrifuge tube and centrifuged (at 3500 rpm) for 2 min. After centrifugation, 100 μL centrifugate was collected to a preweighed counting tube and directly poured into a maintained medium. Cell pellets were frozen with dry ice, and further collected into another weighed counting tube. The weight of the cell pellet and the medium were measured, and radioactivity was determined using a γ scintilation counter (1470 WIZARD Gamma Counter, Wallac, Finland) and normalized to weight. Accumulation of activity of radioactive trancer in cell in vitro is represented by a radio of cell to medium:

$$Ratio of cell to medium = \frac{Radioactivity of cell pellet / Net weight of cell pellet}{Radioactivity of medium / Net weight of medium}$$

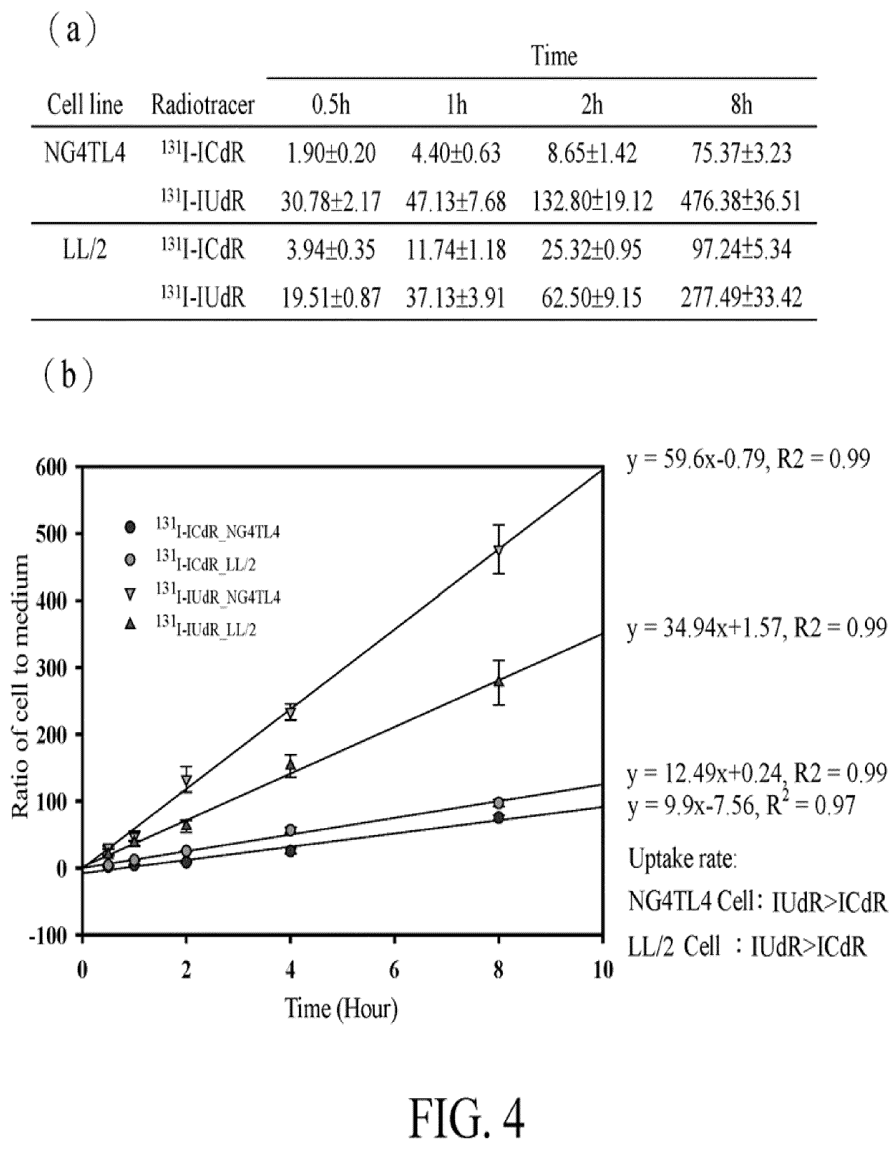

Uptake experimental results of the two radioactive nucleoside analogues [¹³¹I]IUdR and [¹³¹I]ICdR in cells are as shown in FIGS. 4((a), (b)). It is shown that accumulations of ¹³¹I-IUdR in NG4TL4 and LL/2 cells are both higher than those of ¹³¹I-ICdR, while the uptake value of the two are continuously increased with time.

### EXAMPLE 5

### Study on Incorporation in DNA

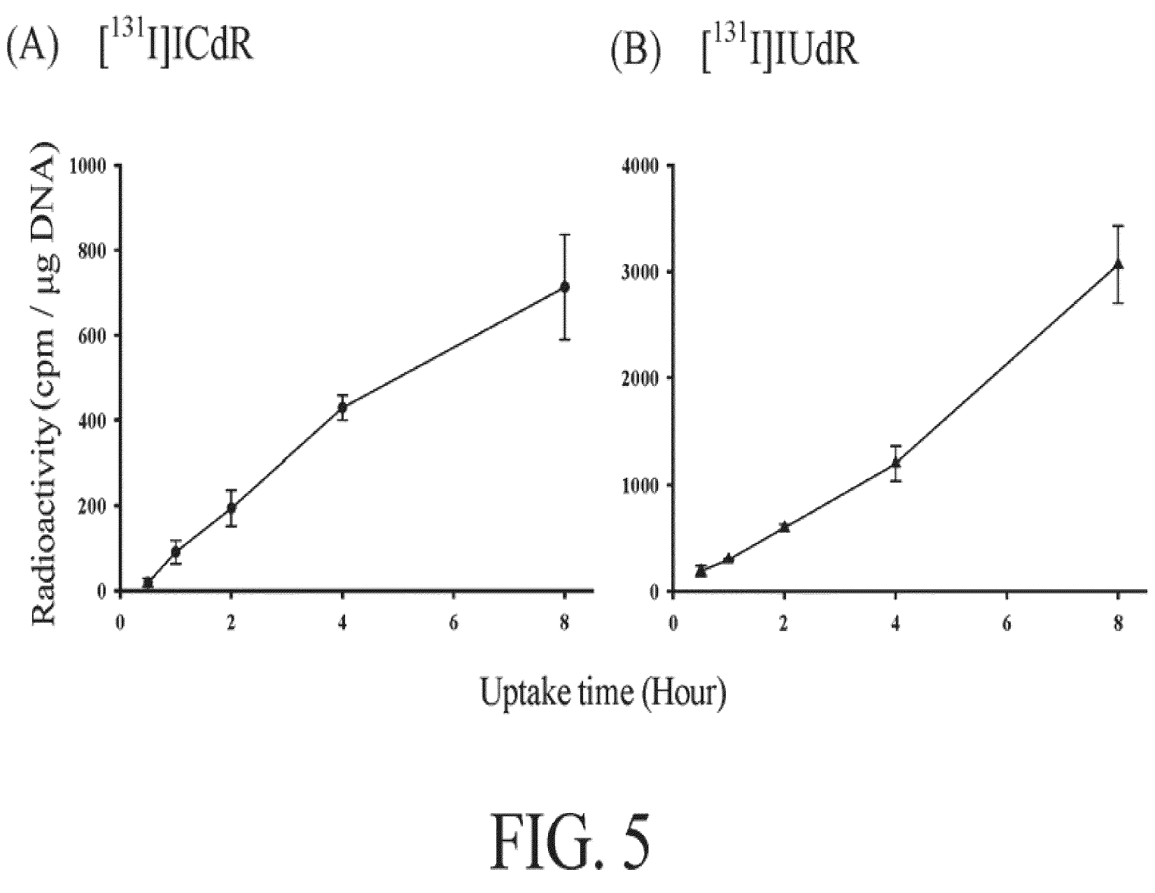

DNA was extracted with Genomic DNA Mini Kit (Geneaid Biotech Ltd., Taiwan). NG4TL4 cell lines were harvested and inoculated into a petri dish at 2×10⁶. After 48-h incubation, the medium in the petri dish was replaced with a serum free medium containig radioactive tracers ¹³¹I-ICdR and ¹³¹I-IUdR (1 μCi/mL medium), and placed in an thermostat incubator at 37° C. At 0.5, 1, 2, 4 and 8 h after incubation, the medium was removed, and the cells was washed two times with ice-cold PBS, and then sheared with trypsin and collected to a centrifuge tube for centrigugation (at 34000 rpm for 1 min). The supernatant was removed, while 50 μL remaining buffer was left to keep the cells in suspension. 300 μL cell lysis buffer was added to the sample and uniformly mixed, the cells was cultured in a water bath at 60° C. for a sereral minutes till the solution was transparent (the sample was inversed once every 3 min). 2 μL RNAse (25 mg/mL) was added to the sample and uniformly mixed, after 5-min incubation at room temperature, 100 μL protein removal buffer was added to the sample, uniformly mixed immediately, and centrifuged for 3 min at full speed (14000 rpm) after 5-min incubation on ice. The resulting solution was transferred as a suspension to another tube, and isopropanol was added and thoroughly mixed. After 20-min centrifugation at full speed (14000 rpm), the suspension was removed, 1 ml dddH₂O was added, and then DNA was lysed for 30 min in a water bath at 60° C. Finally, DNA content was determined with a multimode microplate readers (Infinite®200), and the radioactivity of all samples was measured with a γ counter (1470 WIZARD Gamma Counter, Wallac, Finland), and normalized to DNA weight. In the test, DNA purity was determined at absorption wavelengths of 260 nm and 280 nm, and the ratio of absorbances (OD₂₆₀/OD₂₈₀) was about 1.7. DNA extraction experimental results are as shown in FIGS. 5((a), (b)) and it is found that the ratio of cell to medium (C/M) highly correlates to the DNA aggregation activity (cpm/μg DNA) (r²>0.90).

### EXAMPLE 6

### Analysis of Metabolite

Healthy FVB/N mice (female) were injected with ¹³¹I-ICdR and ¹³¹I-IUdR at tail veil of 9.25 MB q, and scarified by cervical dislocation at different time after administration (at 0.25, 1, and 2 h for ¹³¹I-ICdR; and at 5 and 15 min for ¹³¹I-IUdR). Radioactive metabolites of ¹³¹I-IcdR and ¹³¹I-IudR in blood and urine were analyzed by normal TLC (developing conditions: ¹³¹I-ICdR: ethyl acetate/ethanol=5/1; and ¹³¹I-IUdR: methanol/dichloromethane=1/15) were evaluated. Blood samples were obtained by heart puncture, and then centrifuged at 13,000 rpm for 10 min. Then, the supernatant (about 300 μL) was placed in a 1.5 mL centrifuge tube containing equal amount of ethanol, and then centrifuged again to obtain the serum. The experimental results are as shown in Tables 1 and 2 below.

TABLE 1
Analysis of metabolites in blood and urine through NP-TLC
(developing phase condition: EA/EtOH = 5/1) after ¹³¹I-ICdR
was injected to healthy FVB/N mice (n = 3) at tail vein

| | Species | 0.25 h | 1 h | 2 h |
|---|---|---|---|---|
| Blood | ¹³¹I-ICdR (%) | 63.10 + 1.17 | 72.16 + 3.66 | 67.77 + 7.33 |
| | ¹³¹I-IUdR (%) | 18.62 + 2.25 | 0 | 0 |
| | ¹³¹I-I⁻(%) | 18.28 + 3.39 | 14.11 + 6.15 | 19 + 5.90 |
| | ¹³¹I-compound^{a} (%) | 0 | 13.73 + 1.38 | 13.23 + 1.97 |
| Urine | ¹³¹I-ICdR (%) | 56.49 + 6.58 | 71 + 0.26 | 81.1 + 8.86 |
| | ¹³¹I-IUdR (%) | 3.19 + 1.03 | 0 | 0 |
| | ¹³¹I-I⁻(%) | 40.32 + 6.18 | 26.54 + 0.34 | 15.37 + 7.67 |
| | ¹³¹I-compound^{a} (%) | 0 | 2.46 + 0.08 | 3.53 + 1.96 |

^{a}denotes that the compound is unknown.

TABLE 1
Analysis of metabolites in blood and urine through NP-TLC
(developing phase condition: CH₂Cl₂/MeOH = 15/1) after
¹³¹I-IUdR was injected to healthy FVB/N mice (n = 3) at tail vein

| | Species | 5 min | 15 min |
|---|---|---|---|
| Blood | ¹³¹I-IUdR (%) | 25.23 + 2.88 | 6.42 + 3.89 |
| | ¹³¹I-IU (%) | 3.65 + 3.03 | 1.58 + 0.72 |
| | ¹³¹I-I⁻(%) | 71.12 + 5.83 | 92.00 + 4.51 |
| Urine | ¹³¹I-IUdR (%) | 8.60 + 2.88 | 5.23 + 2.05 |
| | ¹³¹I-IU (%) | 4.45 + 3.03 | 2.76 + 1.23 |
| | ¹³¹I-I⁻(%) | 86.96 + 5.83 | 92.02 + 3.27 |

### EXAMPLE 7

### Pharmacokinetic Study of ¹³¹I-ICdR and ¹³¹I-IUdR

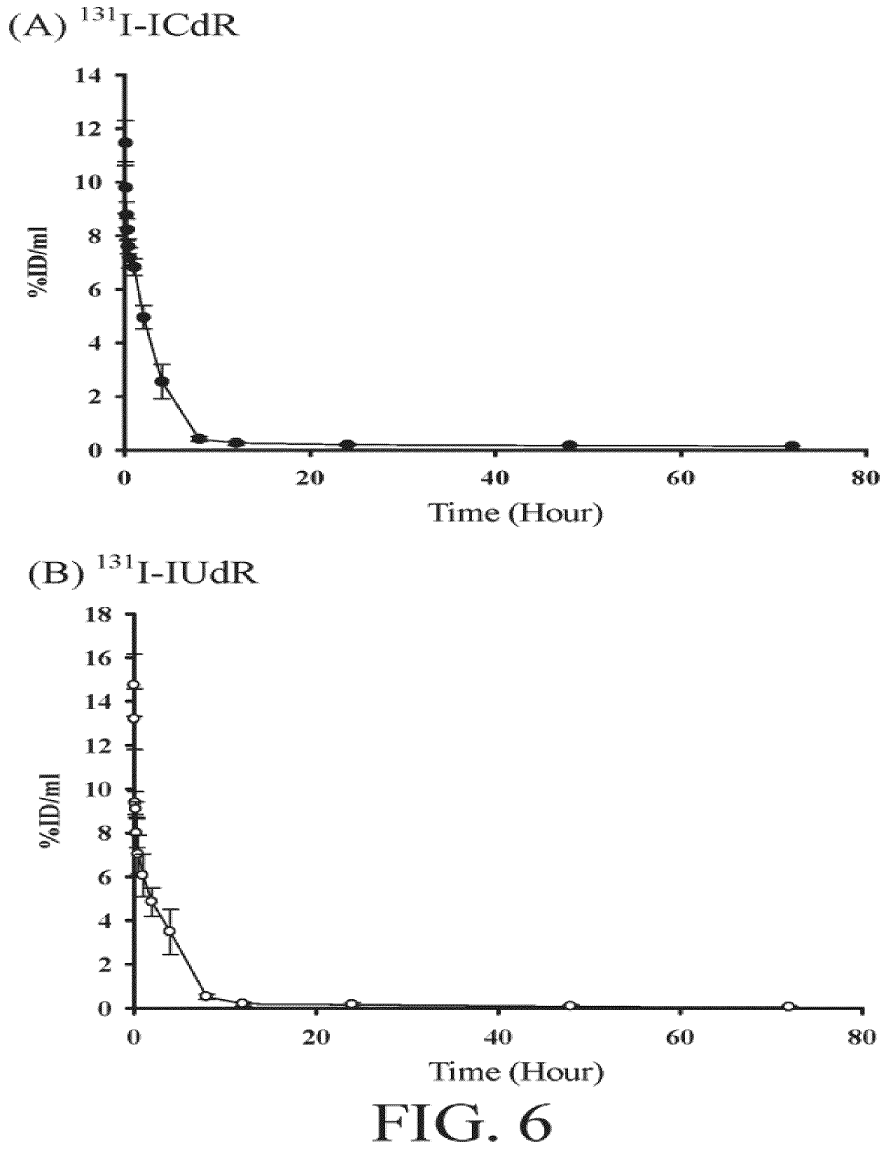

Healthy FVB/N mice (femal) were intravenously injected with 200 μCi ¹³¹I-IcdR or ¹³¹I-IUdR, and then blood samples (with a volume of 1 μL) were collected from lateral tail veil with a quantitative micro capillary (Bluebrand intraEND, Germany) at different time points (at 3, 5, 10, 15, 20 and 30 min, and 1, 2, 4, 8, 12, 24, 48, and 72 h). The radioactivity of the blood samples was measured with a γ counter (1470 WIZARD Gamma Counter, Wallac, Finland) and normalized to blood volume. The concentration of the radioactive compound in the blood was expressed as percentages of ratioactive dosage per milimeter (% ID/mL). Pharmacokinetic parameters were calculated by computer Software WinNonlin 5.2 (Pharsight, Mountain View, Calif., USA). Using two-compartmental analysis model, the calculated paramters included α half life (t_{1/2}α), β half life (t_{1/2}β), Cₘₐₓ, total body clearance and area under curve (AUC). After intravenously injecting ¹³¹I-ICdR or ¹³¹I-IUdR into healthy FVB/N mice, the curve of activity concentration in blood vs time meets two-compartmental analysis model of pharmacokinetics. All parameters were calculated using Software WinNonlin and the pharmacokinetic parameters were summarized in Table 3. The maximal concentrations (Cmax) of ¹³¹I-IcdR and ¹³¹I-IudR in blood were measured to be 9.95±0.71% ID/mL and 18.91±6.16% ID/mL, which were also T max in blood. After intravenous injection, t_{1/2}α and t_{1/2}β of ¹³¹I-ICdR were respectively 1.54±0.47 h and 56.36±9.38 h, indicating that radioactivity of ¹³¹I-IcdR in blood was slowly lowered, and the results indicated that the circulation time of ¹³¹I-IcdR in body was longer than that of ¹³¹I-IudR (t_{1/2}α and t_{1/2}β were 0.08±0.02 h and 2.28±0.90 h). Furthermore, AUC of ¹³¹I-IcdR (45.82±3.57 h×% ID/mL) was greater than that of ¹³¹I-IudR (32.98±5.39 h×% ID/mL), and total body clearance of ¹³¹I-IcdR (3.90±0.59 mL/h) was lower than that of ¹³¹I-IudR (6.04±1.01 mL/h). The experimental results are as shown in Table 3 below and FIGS. 6((a), (b)).

TABLE 3
Evaluation of pharmacokinetic parameters after healthy FVB/N mice
(femal) were injected with ¹³¹I-ICdR and ¹³¹I-IUdR at tail veil

| Parameter | Unit | ¹³¹I-ICdR | ¹³¹I-IUdR |
|---|---|---|---|
| t_{1/2}α | h | 1.54 ± 0.47 | 0.08 ± 0.02 |
| t_{1/2}β | h | 56.36 ± 9.38 | 2.28 ± 0.90 |
| CL | mL/h | 3.90 ± 0.59 | 6.04 ± 1.01 |
| Cₘₐₓ | % ID/mL | 9.95 ± 0.71 | 18.91 ± 6.16 |
| AUC_{0→t} | h × % ID/ | 45.82 ± 3.57 | 32.98 ± 5.39 |
| | mL | | |

It is found through metabolite analysis and pharmacokinetic experimental results that in blood and urine of mice administrated with ¹³¹I-ICdR and ¹³¹I-ICdR is still a main component (at 1 h after administration, concentrations in blood and urine are 72.2% and 71.0% respectively), and ¹³¹I-IUdR is substantially metabolized into free ¹³¹I⁻ (concentrations in blood and urine are 71.1% and 88.0% respectively) after 5 min. Moreover, blood retention time of ¹³¹I-ICdR is longer, suggesting that accumulation of ¹³¹I-ICdR in tumor is more beneficial.

### EXAMPLE 8

### Biodistribution Study of ¹³¹I-ICdR and ¹³¹I-IUdR

FVB/N mice implanted with NG4TL4-WT tumor were injected with radioactive tracers at tail vein, and then scarified by cervical dislocation at specified time points (after 1, 2, 4, and 8 h). Tumor and 13 other tissures (blood, heart, lung, liver, stomach, small intestine, large intestine, spleen, pancreas, kidney, bone, marrow, and muscle) were removed, rinsed, weighed, and determined for radioactivity with a γ scintilation counter. Uptake of the radioactive trancers in the tissues (counts per min) was calibrated against decay, normalized to sample weight, and expressed as percentages of injected dosage per gram of tissue (% ID/g) and aggregation ratio of tumor to blood. The results are as shown in Tables 4 and 5.

TABLE 4
Biodistribution of 80~90 μCi ¹³¹I-ICdR injected into FVB/N mice at tail
veil

| Organ | 1 h | 2 h | 4 h | 8 h |
|---|---|---|---|---|
| Blood | 6.21 + 1.11 | 3.76 + 0.13 | 3.40 + 0.15 | 0.77 + 0.10 |
| Heart | 1.45 + 0.14 | 1.14 + 0.01 | 0.84 + 0.07 | 0.19 + 0.03 |
| Lung | 3.52 + 0.48 | 2.72 + 0.17 | 2.28 + 0.12 | 0.69 + 0.16 |
| Liver | 2.05 + 0.20 | 1.43 + 0.09 | 0.85 + 0.01 | 0.38 + 0.02 |
| Stomach | 17.07 + 1.28 | 14.99 + 4.04 | 17.68 + 0.74 | 2.85 + 0.61 |
| Small | 4.79 + 0.41 | 4.79 + 0.45 | 5.27 + 0.39 | 2.06 + 0.13 |
| Intestine | | | | |
| Large | 2.92 + 0.18 | 2.54 + 0.33 | 2.88 + 0.21 | 1.33 + 0.06 |
| Intestine | | | | |
| Spleen | 4.03 + 0.55 | 3.07 + 0.09 | 2.88 + 0.13 | 2.77 + 0.09 |
| Pancreas | 2.81 + 0.42 | 2.08 + 0.08 | 2.09 + 0.13 | 0.38 + 0.05 |
| Kidney | 3.96 + 0.39 | 2.36 + 0.06 | 2.07 + 0.10 | 0.75 + 0.02 |
| Muscle | 0.85 + 0.04 | 0.61 + 0.05 | 0.51 + 0.04 | 0.09 + 0.01 |
| Tumor | 3.46 + 0.07 | 3.78 + 0.07 | 4.85 + 0.17 | 2.32 + 0.27 |
| Bone | 0.96 + 0.13 | 0.71 + 0.19 | 0.77 + 0.07 | 0.11 + 0.02 |
| Marrow | 3.29 + 0.08 | 3.58 + 1.12 | 3.82 + 0.54 | 2.31 + 0.10 |
| Brain | 0.25 + 0.03 | 0.15 + 0.03 | 0.12 + 0.02 | 0.01 + 0.00 |
| T/M | 4.07 | 6.16 | 9.59 | 25.77 |
| T/B | 0.56 | 1.00 | 1.43 | 3.02 |

TABLE 4
Biodistribution of 80~90 μCi ¹³¹I-IUdR injected into FVB/N mice at tail veil

| Organ | 5 min | 30 min | 1 h | 2 h | 4 h | 8 h |
|---|---|---|---|---|---|---|
| Blood | 13.62 ± 1.19 | 835 ± 132 | 6.41 ± 036 | 4.19 + 0.60 | 1.96 ± 0.49 | 0.48 ± 0.23 |
| Heart | 6J26 ± 0.57 | 3.15 ± 0.4S | 2.43 ± 0.56 | 1.43 ± 0:29 | 0.88 ± 0.46 | 0.19 ± 0.09 |
| Lung | 933 + 0.69 | 5.80 + 0.42 | 4.71 + 1:28 | 2.86 + 0.55 | 1.60 + 0.71 | 0.44 + 0.21 |
| Liver | 15.99 + 2.06 | 3.73 ± 0.57 | 2.68 ± 0.48 | 1.67 ± 0.18 | 1.03 ± O39 | O30 + O.13 |
| Stomach | 7.04 ± 0.98 | 24.71 + 3.20 | 6.18 ± 1.51 | 15.93 + 333 | 4.49 + 3.02 | 0:90 + 0.27 |
| Small Intestine | 10.96 + O.66 | 9.86 + 2:28 | 6.80 + 0.81 | 6.77 ± 1.50 | 4.76 ± 1.26 | 3.40 + 0.60 |
| Large Intestine | 7.18 ± 038 | 5.43 ± 0.88 | 4.73 ± 1.16 | 3.17 ± 0:26 | 2.07 ± O.67 | 1.78 ± 0.57 |
| Spleen | 632 ± 0:27 | 6.79 ± 1.66 | 4.18 ± 035 | 3.60 + 0.40 | 1.86 ± 0.63 | 1.45 ± O.SO |
| Pancreas | 6.43 ± 0.41 | 5.52 ± 0.84 | 2.84 ± 0.57 | 2.50 + 0.58 | 1.18 ± 0.56 | 0:25 ± 0.14 |
| Kidney | 18.81 + 2.44 | 6.95 ± 0.77 | 4.67 ± 0J9I | 2:99 + 0.44 | 1.77 ± 0.71 | 0.56 ± 0.21 |
| Muscle | 3.80 + 0:27 | 1.45 ± 0:20 | 133 ± 0:20 | 0.93 ± 0.14 | 0.53 ± 0.26 | 0.13 ± 0.03 |
| Tumor | 635 ± 1.93 | 6.11 ± 0.97 | 6.61 ± 033 | 5.63 ± 0.74 | 3.97 ± 1.00 | 2.50 + 0.79 |
| Bone | 233 ± 035 | 2.55 ± 030 | 1.59 ± 039 | 132 ± 033 | 0.62 ± 0.29 | 036 ± 0.12 |
| Marrow | 3.98 ± 1.44 | 538 ± 1.57 | 9.86 ± 4.50 | 6.88 ± 1.19 | 6.63 + 3.84 | 5.00 + 1.59 |
| Brain | 0.51 ± 0.0S | 0.50 + 0.12 | 034 ± 0.07 | 0.17 ± 0.02 | 0.13 ± 0.06 | 0.03 ± 0.01 |
| T/M | 1.67 | 4.21 | 4.97 | 6.06 | 7.49 | 19.91 |
| T/B | 0.47 | 0.73 | 1.03 | 134 | 2.03 | 5.17 |

### EXAMPLE 9

### Study of Planar γ and Animal Micro-SPECT/CT Images

Planar γ images were obtained with a dual-head γ-camera (ECAM; Siemens) equipped with a pinhole collimator. 7.4±0.1 MBq ¹³¹I-ICdR and ¹³¹I-IudR were injected into mice at tail vein, and static scan imaging was implemented for 15 min at 1, 2, 4, and 8 h after administration.

SPECT images and CT images were obtained by using an animal micro-SPECT/CT scanner (FLEX Triumph Regular FLEX X-O CT, SPECT CZT 3Head System, GE Healthcare, Northridge, Calif., USA). ¹²³I-ICdR(18.5 MBq) was injected into FVB/N mice bearing NG4TL4-W sarcoma and mice bearing malignant LL/2 lung sarcoma at tail vein. Then, after 2 and 4 hours, the animals were imaged at prone position parallel to a major axis of the scanner for imaging while being anaesthetised by inhalation of oxygen at a flow rate of 2 L/min (containing 2% isoflurane). After gathering the SPECT images, CT images (energy: 80 kVp, 90 μA, 512 projection) were captured, whereas the SPECT images were captured using a low-energy and high-resolution parallel-hole collimator. In vivo imaging were captured with a field of view (FOV) of 120 mm², and the radius of rotation (ROR) is set to be 120 mm, and were processed by a means of filtered back projection using hamming filter (0.54). Animal micro-SPECT images were recreated to an image size of 80×80×80 pixels, CT images were recreated to an image size (pixels) of 512×512×512, and then a means of co-registration is used for co-registering the animal micro-SPECT images and animal micro-CT images using Amira Software (version 4.1.1).

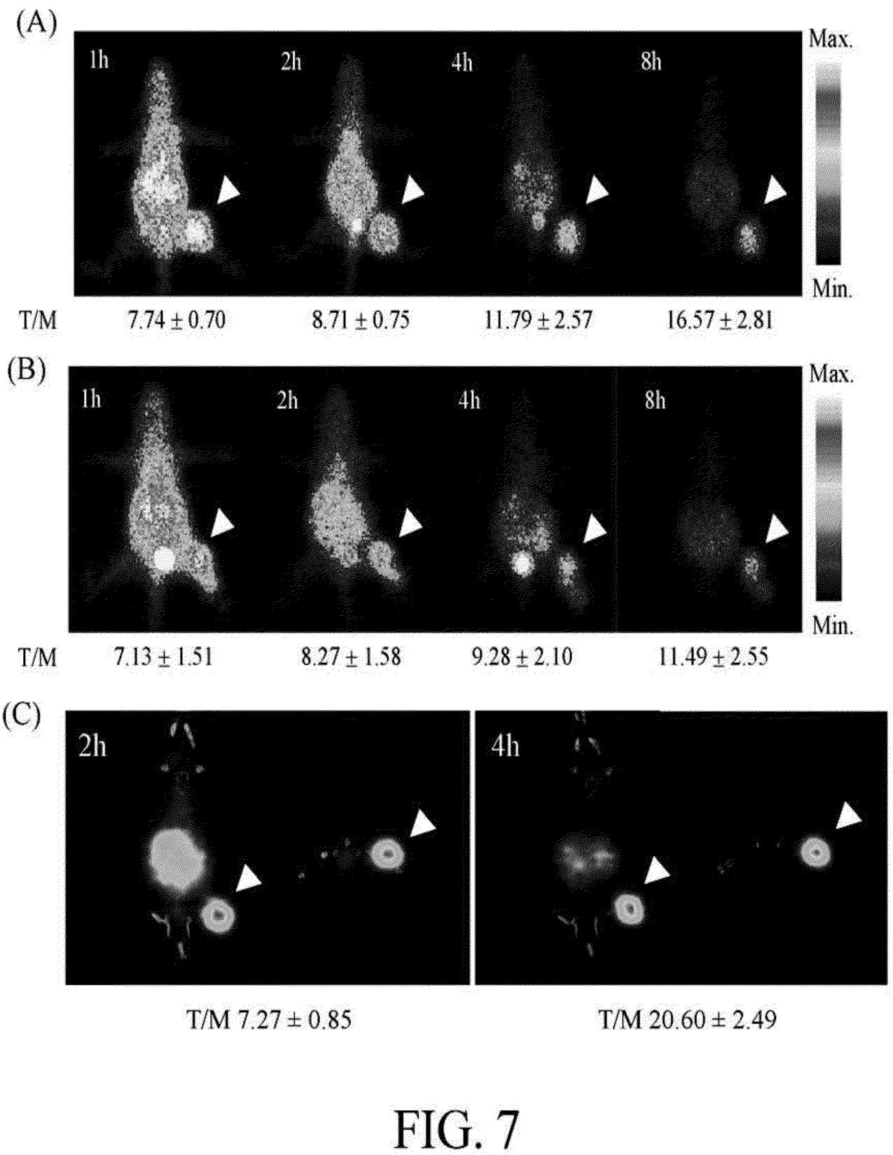

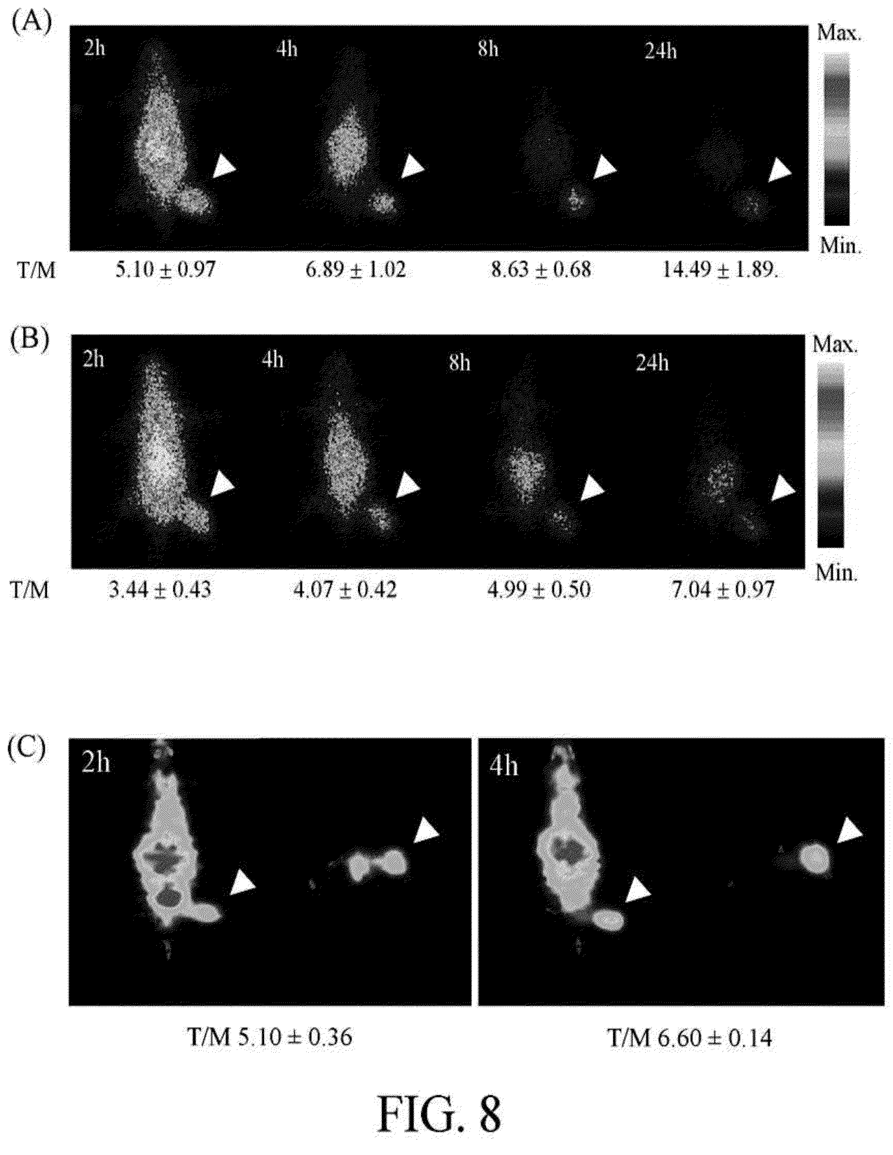

In order to estimate the radioactive concentration, a region of interest covering the tumor and the reference tissue (that is, muscle) were encircled while utilizing the a background of low radioactivity for calibrating the radioactive concentration as the radioactive concentration was measured and obtained at a region far away from the animal body. The radioactive concentration in tumor were normalized to the radioactive concentration in muscle, and expressed as tumor-muscle aggregation ratio (T/M value). The experimental results are as shown in FIGS. 7 and 8((a), (b) and (c)).

Biodistribution and imaging experimental results show that ¹³¹I-ICdR and ¹³¹I-IUdR are obviously accumulated in organs that rapidly proliferates, such as, tumor, marrow, or small intestine, and it is found through biodistribution experimental results that T/M value increases with time, and is 25.77 and 19.91 respectively at the time point of 8 hours. Excretion of the two drugs and metabolites thereof are mainly through the urinary system.

Conclusions: according to the above examples, the present invention has successfully established a radiolabeled nucleoside analogue, and synthesis and analysis of standards thereof. The radiolabeled nucleoside analogue is proved to be suitable for serving as a contrast medium for imaging of tumor proliferation through scintilation planar γ imaging and biodistribution, and can assist in development of imaging in nuclear medicine in tumor detection or therapy prognosis evaluation.

The embodiments are described with examples merely for purpose of easy illustration, and right scope claimed by the present invention is as defined by accompanying claims, but not limited to the embodiment above.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. A radiolabeled nucleoside analogue, comprising a compound having a chemical formula below:
A—B
wherein A is a radioactive ioding comprising ¹²³I, ¹³¹I, and ¹²⁴I, and
B is a pyrimidine derivative selected from a group consisting of cytidine, thymidine, uridine, and a derivative thereof.

2. The radiolabeled nucleoside analogue according to claim 1, wherein the pyrimidine derivative is cytidine or thymidine.

3. The radiolabeled nucleoside analogue according to claim 1, wherein the pyrimidine derivative is a pyrimidine derivative comprising 1-(2-deoxy-β-D-arabinofuranosyl)-5-tributylstannyl.

4. A method for preparing a radiolabeled nucleoside analogue, comprising:
(a) preparing a labeling precursor comprising 5-tributylstannyl-2′-pyrimidine derivative, wherein the pyrimidine derivative in the labeling precursor is selected from a group consisting of cytidine, thymidine, uridine, and a derivative thereof;
(b) iododestannylating the labeling precursor with a radionuclide under an oxidation condition, to obtain radioactive iodine labeled crude product, wherein the radioactive iodine comprises ¹²³I and ¹³¹I; and
(c) purifying the radioactive iodine labeled crude product, to obtain the radiolabeled nucleoside analogue comprising a compound having a chemical formula below:
A—B
wherein A is a radioactive ioding comprising ¹²³I, ¹³¹I, and ¹²⁴I, and
B is a pyrimidine derivative selected from a group consisting of cytidine, thymidine, uridine, and a derivative thereof.

5. The preparation method according to claim 4, wherein the pyrimidine derivative in Step (a) is cytidine or thymidine.

6. The preparation method according to claim 4, wherein the labeling precursor in Step (a) is a pyrimidine derivative comprising 1-(2-deoxy-β-D-arabinofuranosyl)-5-tributylstannyl.

7. The preparation method according to claim 4, wherein the oxidation condition in Step (b) is oxidation with hydrogen peroxide.

8. The preparation method according to claim 4, wherein the purification in Step (c) is performed on a silica gel column.

9. A radiopharmaceutical composition, comprising the radiolabeled nucleoside analogue according to claim 1.

10. The radiopharmaceutical composition according to claim 9, wherein the radiolabeled nucleoside analogue is a compound having a Structural Formula I below:

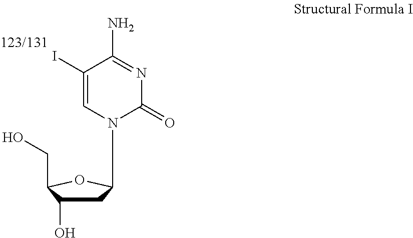

11. The radiolabeled nucleoside analogue according to claim 4, wherein the pyrimidine derivative is cytidine or thymidine.

12. The radiolabeled nucleoside analogue according to claim 4, wherein the pyrimidine derivative is a pyrimidine derivative comprising 1-(2-deoxy-β-D-arabinofuranosyl)-5-tributylstannyl.
